(19) United States
(12) Patent Application Publication
(10) Pub. No.: US 2010/0304448 A1
(43) Pub. Date: Dec. 2, 2010

(54) METHOD FOR THE ENZYMATIC REDUCTION OF ALPHA- AND BETA-DEHYDROAMINO ACIDS USING ENOATE REDUCTASES

(76) Inventors: Rainer Stürmer, Rodersheim-Gronau (DE); Bernhard Hauer, Fussgonheim (DE); Thomas Friedrich, Darmstadt (DE); Kurt Faber, Graz (AT); Melanie Hall, Plouarzel (FR); Clemens Stückler, Graz (AT)

Correspondence Address: CONNOLLY BOVE LODGE & HUTZ, LLP, P O BOX 2207, WILMINGTON, DE, 19899, US

(21) Appl. No.: 12/746,973

(22) PCT Filed: Dec. 8, 2008
(86) PCT No.: PCT/EP08/66977

§ 371 Date: Aug. 18, 2010

(30) Foreign Application Priority Data
Dec. 10, 2007 (EP) 07122758.1

(51) Int. Cl. C12P 13/04

(52) U.S. Cl. 435/106

(57) ABSTRACT

A method for the enzymatic preparation of amino acids of the general formula (3) or (4) from alpha-dehydroamino acids of the general formula (1) or (2)

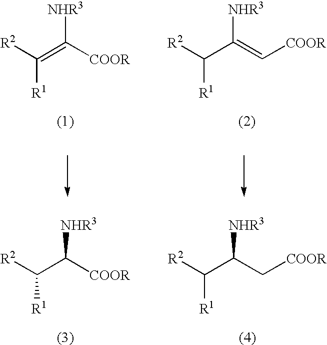

wherein R¹, R² are independently of one another H, C₁-C₆ alkyl, C₂-C₆ alkenyl, an optionally substituted carbo- or heterocyclic, aromatic or nonaromatic radical, or an alkylaryl radical, or a carboxyl radical (—COOR),
R³ is H, formyl, acetyl, propionyl, benzyl, benzyloxycarbonyl, BOC, Alloc,
R is H, C₁-C₆ alkyl, aryl,
   by reducing a compound of the formula (1) or (2) in the presence of a reductase.

### RELATED APPLICATIONS

This application is a national stage application under 35 U.S.C. §371 of PCT/EP2008/066977, filed Dec. 8, 2008, which claims benefit of European application 07122758.1, filed Dec. 10, 2007.

### SUBMISSION OF SEQUENCE LISTING

The Sequence Listing associated with this application is filed in electronic format via EFS-Web and hereby is incorporated by reference in its entirety into the specification. The name of the text file containing the Sequence Listing is Sequence_Listing_{—}12810_{—}01026_US.txt. The size of the text file is 21 KB, and the text file was created on Jun. 8, 2010.

### BACKGROUND OF THE INVENTION

The invention relates to a method for the enzymatic reduction of alpha- and beta-dehydroamino acids of the general formulae (1) and (2).

The object was to provide a method for the enzymatic preparation of compounds of the general formulae (3) and (4), particularly one with a high chemical yield and very good stereoselectivity.

### BRIEF SUMMARY OF THE INVENTION

The above object has been achieved by using the reductases YqjM, OPR1, OPR3 and functional equivalents thereof for reducing alpha-dehydroamino acids of the general formulae (1) and (2).

### DETAILED DESCRIPTION OF THE INVENTION

The invention relates to a method for the enzymatic preparation of amino acids of the general formula (3) or (4) from alpha-dehydroamino acids of the general formula (1) or (2)

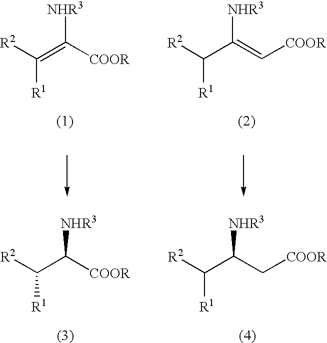

wherein R¹, R² are independently of one another H, C₁-C₆ alkyl, C₂-C₆ alkenyl, an optionally substituted carbo- or heterocyclic, aromatic or nonaromatic radical, or an alkylaryl radical, or a carboxyl radical (—COOR),
R³ is H, formyl, acetyl, propionyl, benzyl, benzyloxycarbonyl, BOC, Alloc,
R is H, C₁-C₆ alkyl, aryl,
by reducing a compound of the formula (1) or (2) in the presence of a reductase
(i) comprising at least one of the polypeptide sequences SEQ ID NO:1, 2, 3, 4, 5, 6, or
(ii) having a functionally equivalent polypeptide sequence which has at least 80% sequence identity with SEQ ID NO:1, 2, 3, 4, 5, 6.

The method of the invention can in principle be carried out both with purified or enriched enzyme itself and with microorganisms which express this enzyme naturally or recombinantly, or with cell homogenates derived therefrom.

Unless stated otherwise, the meanings are:
C₁-C₆-alkyl in particular methyl, ethyl, propyl, butyl, pentyl or hexyl, and the corresponding analogs which are branched one or more times, such as i-propyl, i-butyl, sec-butyl, tert-butyl, i-pentyl or neopentyl, with preference in particular for said C₁-C₄-alkyl radicals;
C₂-C₆-alkenyl in particular the monounsaturated analogs of the abovementioned alkyl radicals having 2 to 6 carbon atoms, with preference in particular for the corresponding C₂-C₄-alkenyl radicals.
Carbo- and heterocyclic aromatic or nonaromatic rings in particular optionally fused rings having 3 to 12 carbon atoms and optionally 1 to 4 heteroatoms such as N, S and O, in particular N or O. Examples which may be mentioned are cyclopropyl, cyclobutyl, cyclopentyl, cyclohexyl, cycloheptyl, the mono- or polyunsaturated analogs thereof such as cyclobutenyl, cyclopentenyl, cyclohexenyl, cycloheptenyl, cyclohexadienyl, cycloheptadienyl; phenyl and naphthyl; and 5- to 7-membered saturated or unsaturated heterocyclic radicals having 1 to 4 heteroatoms which are selected from O, N and S, where the heterocycle may optionally be fused to a further heterocycle or carbocycle. Mention should be made in particular of heterocyclic radicals derived from pyrrolidine, tetrahydrofuran, piperidine, morpholine, pyrrole, furan, thiophene, pyrazole, imidazole, oxazole, thiazole, pyridine, pyran, pyrimidine, pyridazine, pyrazine, coumarone, indole and quinoline. The cyclic radicals, but also the abovementioned alkyl and alkenyl radicals, may optionally be substituted one or more times, such as, for example, 1, 2 or 3 times. Mention should be made as example of suitable substituents of: halogen, in particular F, Cl, Br; —OH, —SH, —NO₂, —NH₃, —SO₃H, C₁-C₄-alkyl and C₂-C₄-alkenyl, C₁-C₄-alkoxy; and hydroxy-C₁-C₄-alkyl; where the alkyl and alkenyl radicals are as defined above, and the alkoxy radicals are derived from the above-defined corresponding alkyl radicals.
BOC is the tert-butoxycarbonyl (protective) group
Alloc is the allyoxylcarbonyl (protective) group

The cyclic radicals listed above may be both carbocycles, i.e. the cycle is composed of carbon atoms only, and heterocycles, i.e. the cycle comprises heteroatoms such as O; S; N. If desired, these carbo- or heterocycles may also additionally be substituted.

The enzymatic reductions of dehydroalanine and dehydroaspartate are particularly advantageous embodiments of the invention.

The reductases suitable for the method of the invention (which are occasionally also referred to as enoate reductases) have a polypeptide sequence as shown in SEQ ID NO:1, 2, or 3 or a polypeptide sequence which has at least 80%, for example at least 90%, or at least 95% and in particular at least 97%, 98% or 99% sequence identity with SEQ ID NO: 1, 2, 3, 4, 5 or 6.

A polypeptide having SEQ ID NO:1 is known as YqjM from Bacillus subtilis. (UniprotKB/Swissprot entry P54550).

A polypeptide having SEQ ID NO:2 is encoded by the tomato OPR1 gene. (UniprotKB/Swissprot entry Q9XG54).

A polypeptide having SEQ ID NO:3 is encoded by the tomato OYPR3 gene (UniprotKB/Swissprot entry Q9FEW9).

A polypeptide having SEQ ID NO:4 is known as Saccharomyces carlsbergensis OYEZ (Genbank Q02899).

A polypeptide having SEQ ID NO:5 is encoded by the OYE2 gene from baker's yeast (Saccharomyces cerevisiae Gene locus YHR179W) (Genbank Q03558).

A polypeptide having SEQ ID NO:6 is encoded by the OYE3 gene from baker's yeast (Saccharomyces cerevisiae Gene locus YPL171C) (Genbank P 41816).

The sequence identity is to be ascertained for the purposes described herein by the “GAP” computer program of the Genetics Computer Group (GCG) of the University of Wisconsin, and the version 10.3 using the standard parameters recommended by GCG is to be employed.

Such reductases can be obtained starting from SEQ ID NO: 1, 2, 3, 4, 5, 6 by targeted or randomized mutagenesis methods known to the skilled worker. An alternative possibility is, however, also to search in microorganisms, preferably in those of the genera Alishewanella, Alterococcus, Aquamonas, Aranicola, Arsenophonus, Azotivirga, Brenneria, Buchnera (aphid P-endosymbionts), Budvicia, Buttiauxella, Candidatus Phlomobacter, Cedecea, Citrobacter, Dickeya, Edwardsiella, Enterobacter, Erwinia, Escherichia, Ewingella, Grimontella, Hafnia, Klebsiella, Kluyvera, Leclercia, Leminorella, Moellerella, Morganella, Obesumbacterium, Pantoea, Pectobacterium, Photorhabdus, Plesiomonas, Pragia, Proteus, Providencia, Rahnella, Raoultella, Salmonella, Samsonia, Serratia, Shigella, Sodalis, Tatumella, Trabulsiella, Wigglesworthia, Xenorhabdus, Yersinia, Yokenella or Zymomonas for reductases which catalyze the abovementioned model reaction and whose amino acid sequence already has the required sequence identity to SEQ ID NO: 1, 2, 3, 4, 5, 6 or is obtained by mutagenesis methods.

The reductase can be used in purified or partly purified form or else in the form of the microorganism itself. Methods for obtaining and purifying dehydrogenases from microorganisms are well known to the skilled worker.

The enantioselective reduction with the reductase preferably takes place in the presence of a suitable cofactor (also referred to as cosubstrate). Cofactors normally used for reduction of the ketone are NADH and/or NADPH. Reductases can in addition be employed as cellular systems which inherently comprise cofactor, or alternative redox mediators can be added (A. Schmidt, F. Hollmann and B. Bühler “Oxidation of Alcohols” in K. Drauz and H. Waldmann, Enzyme Catalysis in Organic Synthesis 2002, Vol. III, 991-1032, Wiley-VCH, Weinheim).

The enantioselective reduction with the reductase additionally preferably takes place in the presence of a suitable reducing agent which regenerates cofactor oxidized during the reduction. Examples of suitable reducing agents are sugars, in particular hexoses such as glucose, mannose, fructose, and/or oxidizable alcohols, especially ethanol, propanol or isopropanol, and formate, phosphite or molecular hydrogen. To oxidize the reducing agent and, associated therewith, to regenerate the coenzyme, it is possible to add a second dehydrogenase such as, for example, glucose dehydrogenase when glucose is used as reducing agent, or formate dehydrogenase when formate is used as reducing agent. This can be employed as free or immobilized enzyme or in the form of free or immobilized cells. Preparation thereof can take place either separately or by coexpression in a (recombinant) reductase strain.

A preferred embodiment of the claimed method is to regenerate the cofactors by an enzymatic system in which a second dehydrogenase, particularly preferably a glucose dehydrogenase, is used.

It may further be expedient to add further additions promoting the reduction, such as, for example, metal salts or chelating agents such as, for example, EDTA.

The reductases used according to the invention can be employed free or immobilized. An immobilized enzyme means an enzyme which is fixed to an inert carrier. Suitable carrier materials and the enzymes immobilized thereon are disclosed in EP-A-1149849, EP-A-1 069 183 and DE-A 100193773, and the references cited therein. The disclosure of these publications in this regard is incorporated in its entirety herein by reference. Suitable carrier materials include for example clays, clay minerals such as kaolinite, diatomaceous earth, perlite, silicon dioxide, aluminum oxide, sodium carbonate, calcium carbonate, cellulose powder, anion exchanger materials, synthetic polymers such as polystyrene, acrylic resins, phenol-formaldehyde resins, polyurethanes and polyolefins such as polyethylene and polypropylene. The carrier materials are normally employed in a finely divided particulate form to prepare the carrier-bound enzymes, with preference for porous forms. The particle size of the carrier material is normally not more than 5 mm, in particular not more than 2 mm (grading curve). It is possible analogously to choose a free or immobilized form on use of the dehydrogenase as whole-cell catalyst. Examples of carrier materials are Ca alginate and carrageenan. Both enzymes and cells can also be crosslinked directly with glutaraldehyde (crosslinking to give CLEAs). Corresponding and further immobilization methods are described for example in J. Lalonde and A. Margolin “Immobilization of Enzymes” in K. Drauz and H. Waldmann, Enzyme Catalysis in Organic Synthesis 2002, Vol. III, 991-1032, Wiley-VCH, Weinheim.

The reaction can be carried out in aqueous or nonaqueous reaction media or in 2-phase systems or (micro)emulsions. The aqueous reaction media are preferably buffered solutions which ordinarily have a pH of from 4 to 8, preferably from 5 to 8. The aqueous solvent may, besides water, additionally comprise at least one alcohol, e.g. ethanol or isopropanol, or dimethyl sulfoxide.

Nonaqueous reaction media mean reaction media which comprise less than 1% by weight, preferably less than 0.5% by weight of water based on the total weight of the liquid reaction medium. The reaction can in particular be carried out in an organic solvent.

Suitable organic solvents are for example aliphatic hydrocarbons, preferably having 5 to 8 carbon atoms, such as pentane, cyclopentane, hexane, cyclohexane, heptane, octane or cyclooctane, halogenated aliphatic hydrocarbons, preferably having one or two carbon atoms, such as dichloromethane, chloroform, tetrachloromethane, dichloroethane or tetrachloroethane, aromatic hydrocarbons such as benzene, toluene, the xylenes, chlorobenzene or dichlorobenzene, aliphatic acyclic and cyclic ethers or alcohols, preferably having 4 to 8 carbon atoms, such as ethanol, isopropanol, diethyl ether, methyl tert-butyl ether, ethyl tert-butyl ether, dipropyl ether, diisopropyl ether, dibutyl ether, tetrahydrofuran or esters such as ethyl acetate or n-butyl acetate or ketones such as methyl isobutyl ketone or dioxane or mixtures thereof. The aforementioned ethers, especially tetrahydrofuran, are particularly preferably used.

The reduction with reductase can for example be carried out in an aqueous organic reaction medium such as, for example, water/isopropanol in any mixing ratio such as, for example, 1:99 to 99:1 or 10:90 to 90:10, or an aqueous reaction medium.

The substrate (1) or (2) is preferably employed in the enzymatic reduction in a concentration from 0.1 g/l to 500 g/l, particularly preferably from 1 g/l to 50 g/l, and can be fed in continuously or discontinuously.

Substrates (1) or (2) may be employed both as E/Z mixtures and as isomerically pure forms.

The enzymatic reduction ordinarily takes place at a reaction temperature below the deactivation temperature of the reductase employed and above −10° C. It is particularly preferably in the range from 0 to 100° C., in particular from 15 to 60° C. and specifically from 20 to 40° C., e.g. at about 30° C.

A possible procedure for example is to mix the substrate (1) or (2) with the reductase, the solvent and, if appropriate, the coenzymes, if appropriate a second dehydrogenase to regenerate the coenzyme and/or further reducing agents, thoroughly, e.g. by stirring or shaking. However, it is also possible to immobilize the reductase in a reactor, for example in a column, and to pass a mixture comprising the substrate and, if appropriate, coenzymes and/or cosubstrates through the reactor. For this purpose it is possible to circulate the mixture through the reactor until the desired conversion is reached.

The reduction is normally carried out until the conversion is at least 70%, particularly preferably at least 85% and in particular at least 95%, based on the substrate present in the mixture. The progress of the reaction, i.e. the sequential reduction of the double bond, can be followed here by conventional methods such as gas chromatography or high pressure liquid chromatography.

“Functional equivalents” or analogs of the specifically disclosed enzymes are, in the context of the present invention, polypeptides which differ therefrom and which still have the desired biological activity such as, for example, substrate specificity. Thus, “functional equivalents” mean for example enzymes which catalyze the model reaction and which have at least 20%, preferably 50%, particularly preferably 75%, very particularly preferably 90% of the activity of an enzyme comprising one of the amino acid sequences listed under SEQ ID NO:1, 2 or 3. Functional equivalents are additionally preferably stable between pH 4 to 10 and advantageously have a pH optimum between pH 5 and 8 and a temperature optimum in the range from 20° C. to 80° C.

“Functional equivalents” also mean according to the invention in particular mutants which have an amino acid other than that specifically mentioned in at least one sequence position of the abovementioned amino acid sequences but nevertheless have one of the abovementioned biological activities. “Functional equivalents” thus comprise the mutants obtainable by one or more amino acid additions, substitutions, deletions and/or inversions, it being possible for said modifications to occur in any sequence position as long as they lead to a mutant having the property profile according to the invention. Functional equivalence also exists in particular when the reactivity patterns agree qualitatively between mutant and unmodified polypeptide, i.e. for example identical substrates are converted at a different rate.

Examples of suitable amino acid substitutions are to be found in the following table:

| Original residue | Substitution examples |
|---|---|
| Ala | Ser |
| Arg | Lys |
| Asn | Gln; His |
| Asp | Glu |
| Cys | Ser |
| Gln | Asn |
| Glu | Asp |
| Gly | Pro |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

“Functional equivalents” in the above sense are also “precursors” of the described polypeptides and “functional derivatives”.

“Precursors” are in this connection natural or synthetic precursors of the polypeptides with or without the desired biological activity.

“Functional derivatives” of polypeptides of the invention can likewise be prepared on functional amino acid side groups or on their N- or C-terminal end with the aid of known techniques. Such derivatives comprise, for example, aliphatic esters of carboxylic acid groups, amides of carboxylic acid groups, obtainable by reaction with ammonia or with a primary or secondary amine; N-acyl derivatives of free amino groups prepared by reaction with acyl groups; or O-acyl derivatives of free hydroxy groups prepared by reaction with acyl groups.

In the case where protein glycosylation is possible, “functional equivalents” of the invention comprise proteins of the type designated above in deglycosylated or glycosylated form, and modified forms obtainable by altering the glycosylation pattern.

“Functional equivalents” of course also comprise polypeptides which are obtainable from other organisms, and naturally occurring variants. For example, it is possible to establish ranges of homologous sequence regions by comparison of sequences, and to ascertain equivalent enzymes based on the specific requirements of the invention.

“Functional equivalents” likewise comprise fragments, preferably individual domains or sequence motifs, of the polypeptides of the invention, which have, for example, the desired biological function.

“Functional equivalents” are additionally fusion proteins which comprise one of the abovementioned polypeptide sequences or functional equivalents derived therefrom and at least one further, heterologous sequence which is functionally different therefrom in its functional N- or C-terminal linkage (i.e. with negligible mutual functional impairment of the parts of the fusion protein). Nonlimiting examples of such heterologous sequences are, for example, signal peptides or enzymes.

Homologs of the proteins of the invention can be identified by screening combinatorial libraries of mutants, such as, for example, truncation mutants. For example, a variegated library of protein variants can be generated by combinatorial mutagenesis at the nucleic acid level, such as, for example, by enzymatic ligation of a mixture of synthetic oligonucleotides. There is a large number of methods which can be used to prepare libraries of potential homologs from a degenerate oligonucleotide sequence. Chemical synthesis of a degenerate gene sequence can be carried out in an automatic DNA synthesizer, and the synthetic gene can then be ligated into a suitable expression vector. Use of a degenerate set of genes makes it possible to provide all the sequences which encode the desired set of potential protein sequences in one mixture. Methods for synthesizing degenerate oligonucleotides are known to the skilled worker (e.g. Narang, S. A. (1983) Tetrahedron 39:3; Itakura et al. (1984) Annu. Rev. Biochem. 53:323; Itakura et al., (1984) Science 198:1056; Ike et al. (1983) Nucleic Acids Res. 11:477).

Several techniques are known in the prior art for screening gene products of combinatorial libraries which have been prepared by point mutations or truncation, and for screening cDNA libraries for gene products having a selected property. These techniques can be adapted to the rapid screening of gene libraries which have been generated by combinatorial mutagenesis of homologs of the invention. The most commonly used techniques for screening large gene libraries, which are subject to high-throughput analysis, include the cloning of the gene library into replicable expression vectors, transformation of suitable cells with the resulting vector library and expression of the combinatorial genes under conditions under which detection of the desired activity facilitates isolation of the vector which encodes the gene whose product has been detected. Recursive ensemble mutagenesis (REM), a technique which increases the frequency of functional mutants in the libraries, can be used in combination with the screening tests to identify homologs (Arkin and Yourvan (1992) PNAS 89:7811-7815; Delgrave et al. (1993) Protein Engineering 6(3):327-331).

The invention further relates to nucleic acid sequences (single- and double-stranded DNA and RNA sequences such as, for example, cDNA and mRNA) which code for an enzyme having reductase activity according to the invention. Nucleic acid sequences which code for example for amino acid sequences shown in SEQ ID NO:1, 2 or 3 or characteristic partial sequences thereof are preferred.

All nucleic acid sequences mentioned herein can be prepared in a manner known per se by chemical synthesis from the nucleotide building blocks, such as, for example, by fragment condensation of individual overlapping complementary nucleic acid building blocks of the double helix. Chemical synthesis of oligonucleotides can take place, for example, in a known manner by the phosphoramidite method (Voet, Voet, 2nd edition, Wiley Press New York, pages 896-897). Addition of synthetic oligonucleotides and filling in of gaps using the Klenow fragment of DNA polymerase and ligation reactions, and general cloning methods are described in Sambrook et al. (1989), Molecular Cloning: A laboratory manual, Cold Spring Harbor Laboratory Press.

Further embodiments for carrying out the enzymatic reduction method of the invention:

The pH in the method of the invention is advantageously kept between pH 4 and 12, preferably between pH 4.5 and 9, particularly preferably between pH 5 and 8. A minimum of 98% ee is achieved.

It is possible to use for the method of the invention growing cells which comprise nucleic acids, nucleic acid constructs or vectors coding for the reductase. It is also possible to use resting or disrupted cells. Disrupted cells mean for example cells which have been made permeable by a treatment with, for example, solvents, or cells which have been disintegrated by an enzyme treatment, by a mechanical treatment (e.g. French press or ultrasound) or by any other method. The crude extracts obtained in this way are advantageously suitable for the method of the invention. Purified or partially purified enzymes can also be used for the method. Immobilized microorganisms or enzymes are likewise suitable and can advantageously be used in the reaction.

The method of the invention can be carried out batchwise, semi-batchwise or continuously.

The method can advantageously be carried out in bioreactors as described, for example, in Biotechnology, Vol. 3, 2nd edition, Rehm et al. editors (1993) especially chapter II.

The products prepared in the method of the invention may be isolated from the reaction medium by methods familiar to the skilled worker and purified, if desired. Said methods include distillation methods, chromatography methods, extraction methods and crystallization methods. The products may be purified to a substantially higher level by combining a plurality of these methods, as required.

The following examples are intended to illustrate the invention without, however, restricting it. Reference is made to the appended figures in this connection.

### EXPERIMENTAL SECTION

### General Protocol Regarding Asymmetric Bioreduction

The asymmetric bioreduction of the substrates was carried out according to the following general protocol using the isolated enzymes YqjM, OPR1, OPR3 and Zymomonas mobilis reductase.

The enzyme preparation (100-200 μg) was added to a solution of the substrate (5 mM) in Tris buffer, 50 mM ph/7.5 (0.8 ml), with the cofactor NADH or NADPH (15 mM), and the reaction was carried out with shaking (140 rpm) at 30° C. After 48 hours, the reaction mixture was extracted with ethyl acetate and the reaction products were analyzed by GC.

The following procedure was chosen when the cofactor recycling system was used:

### NADH/FDH System

To a mixture of substrate (5 mM), oxidized cofactor NAD⁺ (100 μM), ammonium formate (20 mM) in Tris buffer 50 mM pH 7.5 (0.8 ml), FDH (10 u) was added, after the enzyme (100-200 μg) had been added, and the reaction was carried out at 30° C. (140 rpm) for 48 hours.

### NADH/GDH

To a mixture of substrate (5 mM), oxidized cofactor NAD⁺ (100 μM), glucose (20 mM) in Tris buffer 50 mM pH 7.5 (0.8 ml), (D)-GDH (10 u) was added, after the enzyme (100-200 μg) had been added, and the reaction was carried out at 30° C. (140 rpm) for 48 hours.

### NADPH/G6PDH

To a mixture of substrate (5 mM), oxidized cofactor NADP⁺ (10 μM), glucose 6-phosphate (20 mM) in Tris buffer 50 mM pH 7.5 (0.8 ml), G6PDH (10 u) was added, after the enzyme (100-200 μg) had been added, and the reaction was carried out at 30° C. (140 rpm) for 48 hours.

### ADH

An aliquot of OPR1 was added to a Tris-HCl-buffered solution (0.8 ml, 50 mM, pH 7.5) comprising the substrate methyl 2-acetamidoacrylate (5 mM), the cosubstrate 2-propanol (3-60 mM, 0.6-12 mol equivalents) and the oxidized cofactor NAD+ (100 μM). ADH-A was added (approx. 2-3 U), and the mixture was stirred at 120 rpm at 30° C. for 42 h. The product was extracted with ethyl acetate (2×0.5 ml), the combined organic phases were dried over Na₂SO₄, and the samples obtained were analyzed by achiral GC.

ADH_A was expressed in E. coli BL21 (DE3) (vector pETv22b). After a thermal shock at 65° C. for 20 min., the ADH solution was used without any further purification.

An aliquot of the isolated enzyme was added to a Tris-HCl-buffered solution (0.8 ml, 50 mM, pH 7.5) comprising the substrate methyl 2-acetamidoacrylate (5 mM) and the cofactor NADH or NADPH (10 mM). The reaction mixture was stirred at 120 rpm at 30° C. for 64 h. The product was extracted with ethyl acetate (2×0.5 ml), the combined organic phases were dried over Na₂SO₄, and the samples obtained were analyzed by achiral GC.

The product was identified by comparing it with authentic independently synthesized reference material by means of coinjection into GC-MS and achiral GC. Conversion was determined using a 6% cyanopropylphenyl phase capillary column (Varian CP-1301, 30 m, 0.25 mm, 0.25 μm), detector temperature 240° C., injector temperature 250° C., split ratio 30:1. Temperature program for methyl 2-acetamidoacrylate and N-acetyl-alanine methyl ester: 120° C. for 2 min, 10° C./min to 160° C., 30° C./min to 200° C., sustained for 2 min. Retention times: 4.89 min and 5.12 min.

The enantiomeric excess was determined using a modified cyclodextrin capillary column (CHIRALDEX® B-TA, 40 m, 0.25 mm). Detector temperature 200° C., injector temperature 180° C., split ratio 20:1. Temperature program: 130° C. for 5 min, 2° C./min to 135° C., 15° C./min to 180° C., sustained for 2 min. Retention times: (R/S)- and (S/R)-5.18 and 5.35 min, resp. The absolute configuration is “S”, identified by comparison with authentic samples.

TABLE 1
Asymmetric bioreduction of α-dehydroamino acid derivatives using OPR1, OPR3, YqjM, and Zymomonas mobilis reductase

| | | | | | | | | | | Zymomonas | |
|---|---|---|---|---|---|---|---|---|---|---|---|
| | | | | | | | | | | mobilis | |
| | | | | OPR1 | | OPR3 | | YqjM | | Reductase | |
| | Substrate | Product | Cofactor | c. % | E.e. % | c. % | E.e. % | c. % | Ee % | c. % | E.e. % |
| 1 2 3 4 | 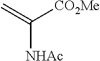 | 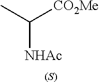 | NADH NADPH NAD⁺/FDH^{a} NADP⁺/G6PDH^{b} | 16 21 63 31 | 91 95 >98 >98 | <1 <1 n.d. n.d. | n.d. n.d. — — | 41 50 91 51 | 97 98 >98 >98 | <1 <1 n.d. n.d. | n.d. n.d. — — |
| 5 6 7 8 | 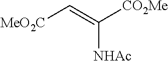 | 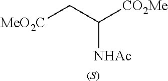 | NADH NADPH NAD⁺/FDH^{a} NADP⁺/G6PDH^{b} | 19 21 98 31 | >99 >99 >99 >99 | <5 <5 n.d. n.d. | n.d. n.d. — — | 26 32 >99 35 | >99 >99 >99 >99 | <5 <5 n.d. n.d. | n.d. n.d. — — |

TABLE 2
Asymmetric bioreduction of α-dehydroamino acid derivatives using OYE1, OYE2 and OYE3

| | | | | OYE1 | | OYE2 | | OYE3 | |
|---|---|---|---|---|---|---|---|---|---|
| | Substrate | Product | Cofactor | c. % | E.e. % | c. % | E.e. % | c. % | E.e. % |
| 1 2 3 4 | 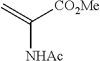 | 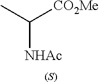 | NADH NADPH NAD⁺/FDH^{a} NADP⁺/G6PDH^{b} | 30 24 7 14 | 95 94 >98 >98 | 6 4 15 9 | 85 74 >98 >98 | 15 17 89 42 | 95 95 >98 >98 |
| 5 6 7 8 | 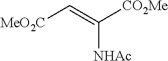 | 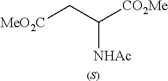 | NADH NADPH NAD⁺/FDH^{a} NADP⁺/G6PDH^{b} | <5 <5 n.d. n.d. | n.d. n.d. — — | n.c. n.c. n.d. n.d. | — — — — | 36 76 >99 49 | 23 10 57 39 |

^{a}PDH = Formate dehydrogenase/format.
^{b}G6PDH = Glucose-6-phosphat dehydrogenase/Glucose-6-phosphate.

TABLE 3

| [2-propanol] mM | Conversion in % |
|---|---|
| 3 (0.6 eq.) | 54 |
| 6 (1.2 eq.) | 90 |
| 7 (1.4 eq.) | >99 |
| 8 (1.6 eq.) | >99 |
| 10 (2 eq.) | >99 |
| 20 (4 eq.) | >99 |
| 60 (12 eq.) | >99 |
| Blank (no OPR1) | <3 |

GC-FID analyses were carried out using a Varian 3800 gas chromatograph with H₂ as carrier gas (14.5 psi).

1. A method for the enzymatic preparation of amino acids of the general formula (3) or (4) from alpha-dehydroamino acids of the general formula (1) or (2) 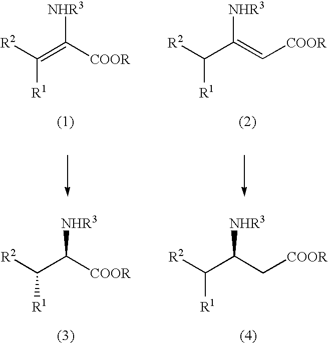
wherein R¹, R² are independently of one another H, C₁-C₆ alkyl, C₂-C₆ alkenyl, an optionally substituted carbo- or heterocyclic, aromatic or nonaromatic radical, or an alkylaryl radical, or a carboxyl radical (—COOR),
R³ is H, formyl, acetyl, propionyl, benzyl, benzyloxycarbonyl, BOC, Alloc,
R is H, C₁-C₆ alkyl, aryl,
by reducing a compound of the formula (1) or (2) in the presence of a reductase
(i) comprising at least one of the polypeptide sequences SEQ ID NO:1, 2, 3, 4, 5, 6, or
(ii) having a functionally equivalent polypeptide sequence which has at least 80% sequence identity with SEQ ID NO:1, 2, 3, 4, 5, 6.

2. The method as claimed in claim 1, wherein the reduction is carried out using NADPH or NADH as cofactor.

3. The method as claimed in claim 2, wherein the cofactor used is enzymatically regenerated.

4. The method as claimed in claim 3, wherein the cofactor is regenerated by glucose dehydrogenase or formate dehydrogenase or a secondary alcohol.

5. The method of claim 1, wherein the reduction is carried out in an aqueous, aqueous-alcoholic or alcoholic reaction medium.

6. The method of claim 1, wherein the reductase is present in an immobilized state.

7. The method of claim 1, wherein the enzyme is selected from among Bacillus subtilis and Lycopersicum esculentum reductases.

8. The method of claim 1, wherein a compound of the formula (1) is reacted, in which
R¹ is H, R² is H, R³ is acetyl.

9. The method of claim 1, wherein the reaction is carried out at a temperature ranging from 0 to 45° C. and/or at a pH ranging from 6 to 8

10. A use of a compound of the formula (3) or (4), prepared by a method of claim 1, as an intermediate for chemical or enzymatic active agent synthesis.


SEQUENCE LISTING (6 sequences)

SEQ ID NO: 1 - protein, 337 aa, Bacillus subtilis
ARKLFTPITIKDMTLKNRIVMSPMCMYSSHEKDGKLTPFHMAHYISRAIGQVGLIIVEASAVNPQGRITDQDLGIWSDEHIEGFAKLTEQVKEQGSKIGIQLAHAGRKAELEGDIFAPSAIAFDEQSATPVEMSAEKVKETVQEFKQAAARAKEAGFDVIEIHAAHGYLIHEFLSPLSNHRTDEYGGSPENRYRFLREIIDEVKQVWDGPLFVRVSASDYTDKGLDIADHIGFAKWMKEQGVDLIDCSSGALVHADINVFPGYQVSFAEKIREQADMATGAVGMITDGSMAEEILQNGRADLIFIGRELLRDPFFARTAAKQLNTEIPAPVQYERGW

SEQ ID NO: 2 - protein, 376 aa, Lycopersicon esculentum
MENKVVEEKQVDKIPLMSPCKMGKFELCHRVVLAPLTRQRSYGYIPQPHAILHYSQRSTNGGLLIGEATVISETGIGYKDVPGIWTKEQVEAWKPIVDAVHAKGGIFFCQIWHVGRVSNKDFQPNGEDPISCTDRGLTPQIRSNGIDIAHFTRPRRLTTDEIPQIVNEFRVAARNAIEAGFDGVEIHGAHGYLIDQFMKDQVNDRSDKYGGSLENRCRFALEIVEAVANEIGSDRVGIRISPFAHYNEAGDTNPTALGLYMVESLNKYDLAYCHVVEPRMKTAWEKIECTESLVPMRKAYKGTFIVAGGYDREDGNRALIEDRADLVAYGRLFISNPDLPKRFELNAPLNKYNRDTFYTSDPIVGYTDYPFLETMT

SEQ ID NO: 3 - protein, 396 aa, Lycopersicon esculentum
MASSAQDGNNPLFSPYKMGKFNLSHRVVLAPMTRCRALNNIPQAALGEYYEQRATAGGFLITEGTMISPTSAGFPHVPGIFTKEQVREWKKIVDVVHAKGAVIFCQLWHVGRASHEVYQPAGAAPISSTEKPISNRWRILMPDGTHGIYPKPRAIGTYEISQVVEDYRRSALNAIEAGFDGIEIHGAHGYLIDQFLKDGINDRTDEYGGSLANRCKFITQVVQAVVSAIGADRVGVRVSPAIDHLDAMDSNPLSLGLAVVERLNKIQLHSGSKLAYLHVTQPRYVAYGQTEAGRLGSEEEEARLMRTLRNAYQGTFICSGGYTRELGIEAVAQGDADLVSYGRLFISNPDLVMRIKLNAPLNKYNRKTFYTQDPVVGYTDYPFLQGNGSNGPLSRL

SEQ ID NO: 4 - protein, 399 aa, Saccharomyces carlsbergensis
SFVKDFKPQALGDTNLFKPIKIGNNELLHRAVIPPLTRMRALHPGNIPNRDWAVEYYTQRAQRPGTMIITEGAFISPQAGGYDNAPGVWSEEQMVEWTKIFNAIHEKKSFVWVQLWVLGWAAFPDNLARDGLRYDSASDNVFMDAEQEAKAKKANNPQHSLTKDEIKQYIKEYVQAAKNSIAAGADGVEIHSANGYLLNQFLDPHSNTRTDEYGGSIENRARFTLEVVDALVEAIGHEKVGLRLSPYGVFNSMSGGAETGIVAQYAYVAGELEKRAKAGKRLAFVHLVEPRVTNPFLTEGEGEYEGGSNDFVYSIWKGPVIRAGNFALHPEVVREEVKDKRTLIGYGRFFISNPDLVDRLEKGLPLNKYDRDTFYQMSAHGYIDYPTYEEALKLGWDKK

SEQ ID NO: 5 - protein, 399 aa, Saccharomyces cerevisiae
PFVKDFKPQALGDTNLFKPIKIGNNELLHRAVIPPLTRMRAQHPGNIPNRDWAVEYYAQRAQRPGTLIITEGTFPSPQSGGYDNAPGIWSEEQIKEWTKIFKAIHENKSFAWVQLWVLGWAAFPDTLARDGLRYDSASDNVYMNAEQEEKAKKANNPQHSITKDEIKQYVKEYVQAAKNSIAAGADGVEIHSANGYLLNQFLDPHSNNRTDEYGGSIENRARFTLEVVDAVVDAIGPEKVGLRLSPYGVFNSMSGGAETGIVAQYAYVLGELERRAKAGKRLAFVHLVEPRVTNPFLTEGEGEYNGGSNKFAYSIWKGPIIRAGNFALHPEVVREEVKDPRTLIGYGRFFISNPDLVDRLEKGLPLNKYDRDTFYKMSAEGYIDYPTYEEALKLGWDKN

SEQ ID NO: 6 - protein, 399 aa, Saccharomyces cerevisiae
PFVKGFEPISLRDTNLFEPIKIGNTQLAHRAVMPPLTRMRATHPGNIPNKEWAAVYYGQRAQRPGTMIITEGTFISPQAGGYDNAPGIWSDEQVAEWKNIFLAIHDCQSFAWVQLWSLGWASFPDVLARDGLRYDCASDRVYMNATLQEKAKDANNLEHSLTKDDIKQYIKDYIHAAKNSIAAGADGVEIHSANGYLLNQFLDPHSNKRTDEYGGTIENRARFTLEVVDALIETIGPERVGLRLSPYGTFNSMSGGAEPGIIAQYSYVLGELEKRAKAGKRLAFVHLVEPRVTDPSLVEGEGEYSEGTNDFAYSIWKGPIIRAGNYALHPEVVREQVKDPRTLIGYGRFFISNPDLVYRLEEGLPLNKYDRSTFYTMSAEGYTDYPTYEEAVDLGWNKN